(19) United States
(12) Patent Application Publication
(10) Pub. No.: US 2006/0240051 A1
(43) Pub. Date: Oct. 26, 2006

(54) Eutectic blends containing a water soluble vitamin derivative

(76) Inventor: Andy Hugh Singleton, Kingsport, TN (US)

Correspondence Address: Eastman Chemical Company, P.O. Box 511, Kingsport, TN, 37662-5075, US

(21) Appl. No.: 11/114,320

(22) Filed: Apr. 26, 2005

(51) Int. Cl. A61K 31/60; A61K 31/192; A61K 9/00

(52) U.S. Cl. 424/400; 514/165; 514/570

(57) ABSTRACT

An eutectic composition having from about 10 to about 90 weight % of a first component comprising a pharmaceutically acceptable substituted C₆-C₁₀ aryl compound wherein the aryl moiety includes a straight or branched moiety selected from the group consisting of C₁-C₁₂ alkyl, C₁-C₁₂ alkoxy, C₂-C₆ alkanoyloxy, hydroxy, carboxy, carboxy substituted C₁-C₁₂ alkyl and mixtures and isomers thereof. The composition also includes from about 10 to 90 weight % of a second component comprising a water-soluble preparation of a fat-soluble vitamin. Also disclosed is a method for preparing the eutectic composition of the present invention. The method includes the steps of forming a mixture from a predetermined amount of the first component with a predetermined amount of the second component, heating the mixture to melt at least one of the components, and mixing the components to form an eutectic blend. In a preferred embodiment, the method further includes the step of cooling the eutectic blend.

### BACKGROUND OF THE INVENTION

The present invention relates to eutectic mixtures or blends containing a water soluble derivative of a fat soluble vitamin, and more particularly eutectic mixtures of a water soluble tocopheryl derivative.

Today, there are many medically useful compounds available to the public by prescription or over-the-counter with many more compounds discovered each year. However, clinical use of these compounds or drugs is possible only if a means for delivering the drug is available. For systemic action, drugs may be orally administered then absorbed in the gastrointestinal tract. Moreover, after a system to deliver the drug is developed and used, skilled drug formulators will often seek to improve or re-design the drug delivery method with the goal to optimize the drug delivery.

The development of an oral dosage form for an active drug is often driven by solubility concerns. The drug formulation, i.e., the combining of the active drug compound together with other inactive compounds and ingredients, will affect the amount or concentration of the drug compound that gets to the active site over the course of a given time period. The composition of the drug formulation directly affects the solubilization of the drug compound in the gastrointestinal tract, and consequently the extent and rate of the absorption of the active drug compound into the blood stream. In addition, the therapeutic value of a drug is also affected by the rate in which the drug dose is released from the delivery system itself, which in turn affects the rate and extent of solubilization of the active compound in the gastrointestinal tract prior to absorption.

Drug compounds of very poor or limited solubility are termed “lipophilic”, “hydrophobic”, or in their most difficult form, “amphiphobic”. A few examples of therapeutic substances in these categories are ibuprofen, diazepam, griseofulvin, cyclosporin, cortisone, proleukin, etoposide and paclitaxel. To increase the solubility of such compounds, and consequently the medicament availability of the drug, skilled formulators have used co-solvents, surfactants or wetting agents to reduce the surface tension of the liquid environment of the gastric fluid in which the active drug is to be dissolved. These agents wet the active drug more quickly so that more of the drug is exposed to the gastric fluid in a shorter time, and may enhance its dissolution. Common types of surfactants and co-solvents that can be used include the cationic, anionic and nonionic types, as well as such co-solvents such as the polyethylene glycols.

A wide variety of lipophilic therapeutic agents are commonly formulated in oil/water emulsion systems. Emulsions and emulsification of a lipophilic drug compound is well known in the medical arts. These conventional emulsions take advantage of the increased solubility of the lipophilic agent in oils such as triglycerides. In their simplest form, an oil-in-water emulsion contains the therapeutic agent solubilized in an oil phase that is dispersed in an aqueous phase with the aid of a surfactant. A recent advance has been the use of α-tocopherol and other tocopherols, tocotrienols or derivatives of these compounds as a solvent to dissolve certain drugs at sufficiently high concentrations to be therapeutically useful.

Vitamin E 1000 TPGS™, or d-α-tocopheryl polyethylene glycol 1000 succinate, has been used as one of many excipients in complex drug formulations, usually emulsions or micro-emulsions, wherein the active drug compound is poorly soluble in water. Vitamin E TPGS™ is a water soluble derivative of Vitamin E in which polyethylene glycol moieties are attached by a succinic acid diester at the ring hydroxyl of the vitamin E molecule. It is recognized that Vitamin E TPGS is a non-ionic surfactant (HLB=16-18) having a dual nature, similar to an amphiphile, that is, hydrophilicity and lipophilicity.

Vitamin E TPGS™ is also believed to enhance bioavailability when co-administered with some lipophilic drugs. Vitamin E TPGS™ is miscible in water and forms solutions with water at concentrations up to approximately 20 weight %. With increasing Vitamin E TPGS™ concentration in water, more complex liquid crystalline phases evolve, e.g., from isotropic globular micellar, to isotropic cylindrical micellar and hexagonal, hexagonal, mixed hexagonal and reversed hexagonal, reversed globular micellar, and to the lamellar phase. It has a melting point of about 38° to 41° C. (100° to 106° F.). It also has a relatively high crystallinity, high degradation temperature, and good thermal stability. A method of making a water soluble derivative from Vitamin E is described in greater detail in U.S. Pat. No. 2,680,749, the disclosure of which is incorporated herein by reference.

U.S. Pat. No. 5,891,845 issued to Myers on Apr. 6, 1999, teaches a solid pharmaceutical composition having from 50 to 99.9 weight % of Vitamin E TPGS and from 0.1 to 50 weight % of a lipophilic drug component. Myers discloses that the advantage of his system is that solid Vitamin E TPGS/drug composition of the present invention does not require the use of surfactants or non-evaporated co-solvents, such as ethanol, because the cyclosporine, or other active drug component, is dissolved by melting the drug directly into Vitamin E TPGS, and is then allowed to cool and harden to form a true molecular solid solution.

U.S. Pat. No. 6,193,985 issued to Sonne on Feb. 27, 2001 discloses an emulsion composition comprising a tocopherol-based phase with an active agent that is sparingly soluble in water with from 20 to 95 weight % of a tocopherol, acetate, linoleate, nicotinate or hemi-succinate derivative that is sufficient to dissolve the active agent in the tocopherol-based phase; and a second phase comprising Vitamin E TPGS™ as an emulsifying agent.

U.S. Pat. No. 6,458,373 issued to Lambert et al. on Oct. 1, 2002 discloses a pharmaceutical composition in an emulsion or microemulsion having an oil and water phases comprising a chemotherapeutic agent, a tocopherol, a tocopherol polyethylene glycol succinate, polyethylene glycol, a surfactant and an aqueous phase. Lambert et al. teach that the pharmaceutical compositions are typically formed by dissolving the therapeutic agent in ethanol to form a therapeutic agent solution. Alpha-tocopherol is then added to the therapeutic agent solution to form an α-tocopherol and therapeutic agent solution. Next, the ethanol is removed from the solution and blended with and without an aqueous phase incorporating a surfactant to form a pre-emulsion. For oral delivery, the pre-emulsion is typically encapsulated in a gelatin capsule.

One advantage that can be obtained by formulating therapeutic agents in liquid form that are then filled into hard shell or soft shell capsules is to provide a more rapid uptake of the therapeutic agent by the gastrointestinal tract due to a short disintegration time of the gelatin capsule within the body.

U.S. Pat. No. 4,690,823 issued to Lohner et al. on Sep. 1, 1987 discloses soft gelatin capsules containing a solution of from 15 to 30 parts by weight of ibuprofen in free acid form (2-(4-isobutylphenyl)propionic acid), in from 70 to 85 parts by weight of polyoxyethylene-polyoxypropylene polymer, or in a mixture of from 30 to 76 parts by weight of polyalkylene glycol and from 7 to 40 parts by weight of a surfactant. Lohner further discloses that up to 3 parts of 1,2-propylene glycol may be added without deteriorating the properties of the ibuprofen. The 1,2-propylene glycol is used easier processability and increase of solubility.

U.S. Pat. No. 6,251,426 issued to Gullapalli on Jun. 26, 2001 discloses a liquid softgel formulation comprising greater than 30% by weight ibuprofen in free acid form in solution; from about 30 to about 60% by weight polyethylene glycol; and from about 10 to about 30% by weight of polyvinylpyrrolidone. The liquid softgel composition is prepared by dissolving more than 30% of ibuprofen in polyethylene glycol and at least 10% by weight of a polyvinylpyrrolidone having an average molecular weight of from about 2,000 to about 54,000. Optionally, the formulations may also include from 1 to about 10 weight % of a surfactant to increase the bioavailability of the ibuprofen. Suitable surfactants include esters of d-α tocopheryl, such as d-α tocopheryl polyethylene glycol 1000 succinate sold by Eastman Chemical Company.

While emulsified and liquid compositions can be prepared by the above methods, one or more of the solvents and/or emulsifying agents may adversely affect gelatin capsules, and particularly softgel capsules, over a period of time. Accordingly, there is still a need for liquid softgel formulations having an effective amount of a pharmaceutically acceptable active agent that do not adversely affect the softgel capsules over a period of time.

### SUMMARY OF THE INVENTION

The present invention is a substantially fluid eutectic composition having from about 10 to about 90-weight % of a first component comprising a pharmaceutically acceptable substituted C₆-C₁₀ aryl compound wherein the aryl moiety includes at least one straight or branched substituent or moiety selected from C₁-C₁₂ alkyl, C₁-C₁₂ alkoxy, hydroxy, C₁-C₆ alkanoyloxy, C₁-C₁₂ carboxy, carboxy substituted C₁-C₁₂ alkyl and mixtures and isomers thereof and from about 10 to 90 weight % of a second component comprising a water-soluble preparation of a fat-soluble vitamin, and preferably is a water-soluble tocopherol ester derivative prepared by esterifying a tocopheryl acid with polyethylene glycol, wherein the weight percentages of the first component and second component equals 100%.

Another aspect of the present invention is a method for preparing the eutectic composition of the present invention. The method comprises the steps of combining a first predetermined amount of the first component with a second predetermined amount of the second component in a suitable container to form a mixture, and mixing the components to form an eutectic blend. In a preferred embodiment, the method further includes the step of heating at least one of the components to a temperature and for a period of time that is sufficient to substantially melt at least one of the components and cooling the eutectic blend.

Advantageously, the eutectic compositions of the present invention are in a substantially fluid state at room temperatures, i.e., temperatures above about 77° F. (25° C.) which allows for ease of handling and filling the softgel capsules.

### DETAILED DESCRIPTION OF THE INVENTION

The present invention relates to a eutectic composition of normally solid compounds. As used herein the term “eutectic composition” or “eutectic mixture” are used interchangeably and mean that the mixture has a melting point lower than the melting point of the constituents composing the mixture, and preferably, the eutectic mixture is a composition comprising greater than about 15 weight % of a fluidic composition after 24 hours and preferably after 48 hours at room temperature and having a viscosity of from about 1000 centipoise (cP) to about 2000 cP as determined using an Advanced Rheometer AR 2000 available from TA instruments and at a temperature of 25° C. Desirably, the eutectic mixture can be used in softgel fill formulations containing high concentrations of a pharmaceutically acceptable compound.

In accordance with the present invention, the eutectic composition has a first component comprising a substituted C₆-C₁₀ aryl compound wherein the aryl moiety includes at least one straight or branched substituent or moiety selected from C₁-C₁₂ alkyl, C₁-C₁₂ alkoxy, hydroxy, C₂-C₆ alkanoyloxy, C₁-C₁₂ carboxy, carboxy substituted C₁-C₁₂ alkyl and mixtures and isomers thereof. Preferably, the substituted aryl compound includes up to four independently selected straight or branched moieties described above. More preferably the aryl compound is di- or tri-substituted with a straight or branched moiety independently selected from C₁-C₆ alkyl, C₁-C₆ alkoxy, C₂-C₃ alkanoyloxy, hydroxy, carboxy, monocarboxy substituted C₁-C₆ alkyl, di-carboxy substituted C₁-C₆ alkyl and mixtures thereof. In preferred embodiment the di-substituted aryl compound includes at least one moiety selected from of a straight or branched: C₁-C₆ carboxy, carboxy substituted C₁-C₆ alkyl, C₁-C₆ acid ester or a C₂-C₃ alkanoyloxy, and the tri-substituted aryl compound includes at least one moiety selected from hydroxy, carboxy, or carboxy substituted C₁-C₆ alkyl. Most preferably the first component is 2-(4-isobutylphenyl)propionic acid, (ibuprofen), butylated hyroxyanisole (BHA), acetyl salicylic acid (aspirin) or a mixture thereof.

The eutectic composition includes a second component comprising a water-soluble preparation of a fat-soluble vitamin. The preferred water-soluble preparation of a fat-soluble vitamin is a water soluble derivative of well known vitamin E-active tocopherols, such as those disclosed in U.S. Pat. No. 2,680,749, the entire disclosure of which is incorporated herein by reference. Generally, the water-soluble tocopherol derivatives are prepared by esterifying a tocopheryl acid ester with polyethylene glycol. The polyoxyethylene glycol moiety has a molecular weight of about 200 to about 20,000, preferably from about 400 to about 2000, more preferably from about 400 to about 1500 and most preferably the water-soluble preparation of a fat-soluble vitamin is Vitamin E polyethylene glycol 1000 succinate available from Eastman Chemical Company under the trade name Vitamin E 1000 TPGS™. Vitamin E 1000 TPGS™ is very stable and does not hydrolyze under normal conditions.

The eutectic mixture of the present invention has from about 10 weight % to about 90 weight % of the first component and from about 90 weight % to about 10 weight % of the second component, preferably from about 20 to about 70 weight % of the first component and from 80 to 30 weight % of the second component, more preferably from about 30 weight % to about 50 weight % of the first component and from about 50 weight % to about 70 weight % of the second component, and most preferably from about 40 weight % to about 50 weight % of the first component and from about 50 weight % to about 60 weight % of the second component, wherein the above percentages are based on the total weight of the first and second components and the total or sum of the percentages is 100%.

Although the above formulations result in a substantially fluidic composition, one skilled in the art will understand that the eutectic composition of the present invention in any given formulation may also have from about 0.1 weight % to about 10 weight %, based on the total weight of the eutectic composition, of other ingredients. Non-limiting examples of such other ingredients include: pseudoephedrine hydrochloride; diphenhydramine hydrochloride; solvents such as polyethylene glycol and polypropylene glycol having molecular weights from about 200 to 2,000 and preferably in a range from between 300 and 630; surfactants such as polyoxyethylene glycerol trihydroxystearate having from 35 to 65 ethylene oxide units, C₁₂-C₁₈ polyoxyethylene fatty alcohol ethers having from 15 to 25 ethylene oxide units, polyoxyethylene stearate having from 15 to 45 ethylene oxide units, C₁₂-C₁₈ polyoxyethylenesorbitan mono fatty acid esters having from 15 to 25 ethylene oxide units, d-α-tocopheryl, and polyoxyethylene castor oil derivatives.

It has been discovered that a eutectic composition can be advantageously prepared using only the two components described above and in the respective weight percentages. It was surprising and unexpectedly discovered that in the case where the alkyl substituted aryl compound is ibuprofen in the free acid state, the presence of greater than about 10 weight % of the second component does not result in a reduction in the amount of ibuprofen in the free acid state. Given the teaching of U.S. Pat. No. 6,251,426, one skilled in the art would have expected a reduction of the free acid due to an expected esterification reaction between the acid and polyethylene glycol. This is quite advantageous since it is well recognized that water-soluble tocopherol derivatives have health advantages and may be utilized with other pharmaceutically acceptable medicaments known to those skilled in the art.

The eutectic compositions of the present invention may be prepared by forming a mixture of the first component with the second component in a suitable container, heating the mixture to a predetermined temperature for a period of time that is sufficient to substantially melt or fluidize at least one of the components, and mixing the mixture sufficiently to form a substantially fluidic eutectic blend. Preferably, the second component is sufficiently melted to disperse, and preferably solubilize, the first component. Desirably, the mixture is heated to a temperature of from about 40° C. to about 95° C., and preferably from about 50° C. to about 80° C. for a period of time ranging from about 1 minute to about 24 hours. After heating, the components are sufficiently mixed using techniques and apparatuses known to those skilled in the art to form a substantially fluidic eutectic blend. After mixing, the eutectic blend can be cooled to room temperature. If it is desired to incorporate optional components such as surfactants, solvents or other medicaments, such optional components may be added to mixture prior to heating or added to the fluidic eutectic blend after heating but preferably before cooling.

Alternatively, the eutectic compositions of the present invention may be prepared by heating the predetermined amount of the second component to a predetermined temperature and for a period of time that is sufficient to substantially melt or fluidize the second component. The first component is then added to the melted second component and mixed sufficiently to form a substantially fluidic eutectic blend. If it is desired to incorporate optional components such as surfactants, solvents or other medicaments, such optional components may be added to the melted second component before, concurrently with, or after the first component is added to the melted second component. The mixture is then mixed sufficiently to form a substantially fluidic eutectic blend. After mixing, the eutectic blend is cooled to room temperature. The eutectic blend may then incorporated in soft gelatin capsules in a known manner.

The eutectic composition of the present invention may be used in formulations for preparing liquid softgel capsules. Accordingly, the eutectic composition is encapsulated into a one-piece gelatin sheath or shell. Various sheath formulations known in the prior art may be used to encapsulate the eutectic formulations of the present invention. For example, suitable sheath formulations may include from about 35 to about 50 weight % gelatin; at least 20 weight %, and preferably up to about 40 weight %, of a plasticizer; and from about 25 to about 50 weight % water. These formulations, when formed into capsules and dried, will result in capsule sheaths comprised of from about 45 to about 75% by weight gelatin; from about 20% to about 40% by weight plasticizer; and from about 5 to about 15% by weight water.

The sheath formulations may also contain other ingredients, such as taste modifiers, coloring agents, and moisture retaining agents. Taste modifiers include up to about 5 weight % of the sheath composition of non-reducing sugars, such as xylitol or maltitol. Suitable moisture retaining agents include cellulose, cellulose derivatives, starch, starch derivatives, vegetable gums, non-hygroscopic, mono-, di- and oligosaccharides, and silicon dioxide. Various FD&C coloring agents may be also be used to impart the desired color to the capsule.

The softgel capsules may be produced in a known manner such as using a rotary die process in which a molten mass of a gelatin sheath formulation is fed from a reservoir onto drums to form two spaced sheets or ribbons of gelatin in a semi-molten state. These ribbons are fed around rollers and brought together at a convergent angle into the nip of a pair of roller dies that include opposed die cavities. A fill formulation to be encapsulated is fed into the wedge-shaped joinder of the ribbons. The gelatin ribbons are continuously conveyed between the dies, with portions of the fill formulation being trapped between the sheets inside the die cavities. The sheets are then pressed together, and severed around each die so that opposed edges of the sheets flow together to form a continuous gelatin sheath around the entrapped medicament. The part of the gelatin sheet that is severed from the segments forming the capsules is then collected for recycling, and the soft capsules are dried.

In order to be acceptable to the consumer, the softgel capsule should be of a size that is easily swallowed. Generally, the fill size of the capsule will be less than 600 mg, and preferably about 500 mg or less, for the capsule to be of an acceptably small dimension. An effective dosage of the active agent, such as ibuprofen, will normally be at least 100 mg to about 175 mg, and preferably about 200 mg. Advantageously, a free flowing, eutectic mixture may be prepared from only the first and second components, allowing the compositions to be free of water and other ingredients that increase the fill volume.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

### EXAMPLE 1

In preparing the sample, 9.8 grams of a water-soluble form of vitamin E (Vitamin E TPGS D-1000 NF, available from Eastman Chemical Company, Kingsport, Tenn.), were weighed into a PYREX® Media bottle and 0.2 grams of butylated hyroxyanisole (BHA, available from Eastman Chemical Company, Kingsport, Tenn.) were then added to the bottle. The bottle was sealed then placed in an oven. The oven temperature was set at 80° C. After six hours the sample was removed and mixed for one minute at a speed of 2200 rpm using a Mini Vortexer MV1, (available from IKA Works, Inc., Wilmington, N.C.)

The sample was returned to the oven. After eighteen hours the oven was turned off. The sample was allowed to cool to room temperature then removed. The blend was a white waxy solid at room temperature.

### EXAMPLE 2

The procedure of Example 1 above was followed except for the following differences: 9.6 grams of Eastman Vitamin E TPGS D-1000 NF were weighed into a PYREX® Media bottle and 0.4 grams of BHA were then added to the bottle. The bottle was sealed then placed in an oven. The blend was a white waxy solid at room temperature.

### EXAMPLE 3

The procedure of Example 1 above was followed except for the following differences: 9.4 grams of Eastman Vitamin E TPGS D-1000 NF were weighed into a PYREX® Media bottle and 0.6 grams of BHA were then added to the bottle. The blend was a white waxy solid at room temperature.

### EXAMPLE 4

The procedure of Example 1 above was followed except for the following differences: 9.2 grams of Eastman Vitamin E TPGS D-1000 NF were weighed into a PYREX® Media bottle and 0.8 grams of BHA were then added to the bottle. The blend was a white waxy solid at room temperature.

### EXAMPLE 5

The procedure of Example 1 above was followed except for the following differences: 9.0 grams of Eastman Vitamin E TPGS D-1000 NF were weighed into a PYREX® Media bottle and 1.0 gram of BHA was then added to the bottle. The blend was a white waxy solid at room temperature.

### EXAMPLE 6

The procedure of Example 1 above was followed except for the following differences: 8.0 grams of Eastman Vitamin E TPGS D-1000 NF were weighed into a PYREX® Media bottle and 2.0 grams of BHA were then added to the bottle. The blend was free flowing with no noticeable haze or clouding, and had a yellowish appearance. After forty-eight hours the sample began to crystallize.

### EXAMPLE 7

The procedure of Example 1 above was followed except for the following differences: 7.5 grams of Eastman Vitamin E TPGS D-1000 NF were weighed into a PYREX® Media bottle and 2.5 grams of BHA were then added to the bottle. The blend was free flowing clear with a yellow appearance. After forty-eight hours the sample began to crystallize.

### EXAMPLE 8

The procedure of Example 1 above was followed except for the following differences: 7.0 grams of Eastman Vitamin E TPGS D-1000 NF were weighed into a PYREX® Media bottle and 3.0 grams of BHA were then added to the bottle. The blend was free flowing, clear, with a yellow appearance. After twenty-seven months the sample was stable and free flowing.

### EXAMPLE 9

The procedure of Example 1 above was followed except for the following differences: 6.0 grams of Eastman Vitamin E TPGS D-1000 NF were weighed into a PYREX® Media bottle and 4.0 grams of BHA were then added to the bottle. The blend was free flowing, clear, with a yellow appearance. After twenty-seven months the sample was stable and free flowing.

### EXAMPLE 10

The procedure of Example 1 above was followed except for the following differences: 5.0 grams of Eastman Vitamin E TPGS D-1000 NF were weighed into a PYREX® Media bottle and 5.0 grams of BHA were then added to the bottle. The blend was free flowing, clear, with a yellow appearance. After twenty-seven months the sample was stable and free flowing.

### EXAMPLE 11

The procedure of Example 1 above was followed except for the following differences: 4.0 grams of Eastman Vitamin E TPGS D-1000 NF were weighed into a PYREX® Media bottle and 6.0 grams of BHA were then added to the bottle. The blend was free flowing, clear, with a yellow appearance. The sample began to crystallize after seventy-two hours.

### EXAMPLE 12

The procedure of Example 1 above was followed except for the following differences: 3.0 grams of Eastman Vitamin E TPGS D-1000 NF were weighed into a PYREX® Media bottle and 7.0 grams of BHA were then added to the bottle. The blend was free flowing, clear, with a yellow appearance. The sample began to crystallize after seventy-two hours.

### EXAMPLE 13

The procedure of Example 1 above was followed except for the following differences: 2.0 grams of Eastman Vitamin E TPGS D-1000 NF were weighed into a PYREX® Media bottle and 8.0 grams of BHA were then added to the bottle. The blend was free flowing, clear, with a yellow appearance. The sample began to crystallize after seventy-two hours.

### EXAMPLE 14

The procedure of Example 1 above was followed except for the following differences: 1.5 grams of Eastman Vitamin E TPGS D-1000 NF were weighed into a PYREX® Media bottle and 8.5 grams of BHA were then added to the bottle. The blend was free flowing, clear, with a yellow appearance. After twenty-four hours the sample began to crystallize. After twenty-seven months the top 25% of the sample was liquid and the bottom 75% was solid.

### EXAMPLE 15

The procedure of Example 1 above was followed except for the following differences: 1.0 gram of Eastman Vitamin E TPGS D-1000 NF was weighed into a PYREX® Media bottle and 9.0 grams of BHA were then added to the bottle. The blend was free flowing, clear, with a yellow appearance. The sample began to crystallize after seventy-two hours.

### EXAMPLE 16

The procedure of Example 1 above was followed except for the following differences: 0.8 grams of Eastman Vitamin E TPGS D-1000 NF were weighed into a PYREX® Media bottle and 9.2 grams of BHA were then added to the bottle. The blend was a white solid at room temperature.

### EXAMPLE 17

The procedure of Example 1 above was followed except for the following differences: 0.6 grams of Eastman Vitamin E TPGS D-1000 NF were weighed into a PYREX® Media bottle and 9.4 grams of BHA were then added to the bottle. The blend was a white solid at room temperature.

### EXAMPLE 18

The procedure of Example 1 above was followed except for the following differences: 0.2 grams of Eastman Vitamin E TPGS D-1000 NF were weighed into a PYREX® Media bottle and 9.8 grams of BHA were then added to the bottle. The blend was a white solid at room temperature.

### EXAMPLE 19

The procedure of Example 1 above was followed except for the following differences: 9.0 grams of Eastman Vitamin E TPGS D-1000 NF were weighed into a PYREX® Media bottle and 1.0 gram of ibuprofen was then added to the bottle. The blend crystallized after cooling to room temperature.

### EXAMPLE 20

The procedure of Example 1 above was followed except for the following differences: 8.0 grams of Eastman Vitamin E TPGS D-1000 NF were weighed into a Pyrex Media bottle and 2.0 grams of ibuprofen were then added to the bottle. The blend was free flowing, clear with a slight yellow appearance. After twelve hours the blend began to crystallize.

### EXAMPLE 21

The procedure of Example 1 above was followed except for the following differences: 7.0 grams of Eastman Vitamin E TPGS D-1000 NF were weighed into a PYREX® Media bottle and 3.0 grams of ibuprofen were then added to the bottle. The blend was free flowing, clear with a slight yellow appearance. After forty-eight hours at room temperature the sample began to crystallize.

### EXAMPLE 22

The procedure of Example 1 above was followed except for the following differences: 6.0 grams of Eastman Vitamin E TPGS D-1000 NF were weighed into a PYREX® Media bottle and 4.0 grams of ibuprofen were then added to the bottle. The blend was free flowing, clear with a slight yellow appearance. After twenty-seven months the sample was stable and free flowing.

### EXAMPLE 23

The procedure of Example 1 above was followed except for the following differences: 5.0 grams of Eastman Vitamin E TPGS D-1000 NF were weighed into a PYREX® Media bottle and 5.0 grams of ibuprofen were then added to the bottle. The blend was free flowing, clear with a slight yellow appearance. After twenty-seven months the sample was stable and free flowing.

### EXAMPLE 24

The procedure of Example 1 above was followed except for the following differences: 4.0 grams of Eastman Vitamin E TPGS D-1000 NF were weighed into a Pyrex Media bottle and 6.0 grams of ibuprofen were then added to the bottle. The blend was free flowing, clear with a slight yellow appearance. After forty-eight hours the sample began to crystallize.

### EXAMPLE 25

The procedure of Example 1 above was followed except for the following differences: 3.0 grams of Eastman Vitamin E TPGS D-1000 NF were weighed into a PYREX® Media bottle and 7.0 grams of ibuprofen were then added to the bottle. The blend was free flowing, clear with a slight yellow appearance. The sample began to crystallize after forty-eight hours.

### EXAMPLE 26

The procedure of Example 1 above was followed except for the following differences: 2.0 grams of Eastman Vitamin E TPGS D-1000 NF were weighed into a PYREX® Media bottle and 8.0 grams of ibuprofen were then added to the bottle. The blend was free flowing, clear with a slight yellow appearance. The sample began to crystallize after forty-eight hours.

### EXAMPLE 27

The procedure of Example 1 above was followed except for the following differences: 1.0 grams of Eastman Vitamin E TPGS D-1000 NF were weighed into a PYREX® Media bottle and 9.0 grams of ibuprofen were then added to the bottle. The blend was free flowing, clear with a slight yellow appearance. The sample began to crystallize after forty-eight hours.

### EXAMPLE 28

The following examples illustrate the effect of water on the eutectic blends of the present invention. The procedure of Example 1 above was followed except for the following differences: 9 grams of Eastman Vitamin E TPGS D-1000 were weighed into a PYREX® Media bottle and 1 gram of nanopure water was then added to the bottle. Upon cooling the blend was free flowing, clear with a slight yellow slight yellow appearance. At this point 0.25 grams of ibuprofen were added to the bottle. The bottle was sealed then placed in an oven. The oven temperature was set at 80° C. After six hours the sample was removed and mixed thoroughly using a vortexer. The sample was returned to the oven and after eighteen hours the oven was turned off. The sample was allowed to cool to room temperature then removed. The blend was free flowing, clear with a slight yellow appearance.

### EXAMPLE 29

In preparing the sample, 8.75 grams of Eastman Vitamin E TPGS D-1000 NF were weighed into a PYREX® Media bottle and 0.25 grams of Ibuprofen were then added to the bottle. The bottle was sealed then placed in an oven. The oven temperature was set at 80° C. After 1 hour the sample was removed and mixed thoroughly using a vortexer. At this point, 1 gram of Nanopure water was added to the bottle. The sample was returned to the oven. After 6 hours the sample was removed and mixed thoroughly using a vortexer. The sample was returned to the oven and after eighteen hours the oven was turned off. The sample was allowed to cool to room temperature then removed. The blend was free flowing, clear with a slight yellow appearance.

### EXAMPLE 30

The procedure of Example 29 above was followed except for the following differences: 8.0 grams of Eastman Vitamin E TPGS D-1000 were weighed into a PYREX® Media bottle and 1.0 gram of Ibuprofen was then added to the bottle. The blend was free flowing, clear with a slight yellow appearance.

### EXAMPLE 31

In preparing the sample, 9.0 grams of Eastman Vitamin E TPGS D-1000 NF were weighed into a PYREX® Media bottle and 1.0 gram of Nanopure water was then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The sample was a solid at room temperature. At this point 1.0 gram of ibuprofen was added to the sample. The heating and mixing procedures were repeated. The sample was allowed to cool to room temperature then removed. The blend was free flowing, clear with a slight yellow appearance. After twenty-nine months the sample was stable and free flowing.

### EXAMPLE 32

In preparing the sample, 8.7 grams of Eastman Vitamin E TPGS D-1000 were weighed into a PYREX® Media bottle and 0.3 grams of PEG 1000 were then added to the bottle. One gram of Nanopure water was then added. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was free flowing, clear with a slight yellow appearance. The additional spiking with PEG 1000 had no effect on lowering the viscosity of the sample.

### EXAMPLE 33

In preparing the sample, eight grams of Eastman Vitamin E TPGS D-1000 were weighed into a PYREX® Media bottle and 2 grams of Nanopure water were then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The sample was a gel at room temperature. At this point 1.0 gram of methyl paraben was added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The sample was a gel at room temperature. The post addition of methyl paraben had no effect on lowering the viscosity of the sample.

### EXAMPLE 34

In preparing the sample, 8 grams of Eastman Vitamin E TPGS D-1000 were weighed into a PYREX® Media bottle and 2 grams of Nanopure water were then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. At this point 1.0 gram of sucrose acetate isobutyrate (SAIB, available from Eastman Chemical Company) was added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. The post addition of SAIB had not effect on lowering the viscosity of the sample.

### EXAMPLE 35

In preparing the sample, 8 grams of Eastman Vitamin E TPGS D-1000 were weighed into a PYREX® Media bottle and 2 grams of Nanopure water were then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. At this point 1.0 gram of caprylic/capric acid triglycerides (available from ABITEC Corporation, Janesville, Wis. under the trade name Captex 300) was added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. The post addition of Captex 300 had no immediate effect on lowering the viscosity of the blend. After twenty-seven months the sample was cloudy, flowing and highly viscous.

### EXAMPLE 36

In preparing the sample, 8 grams of Eastman Vitamin E TPGS D-1000 were weighed into a PYREX® Media bottle and 2 grams of Nanopure water were then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. At this point 1.0 gram of butylated hydroxytoluene (BHT, available from Eastman Chemical Company) was added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. The post addition of BHT had no effect on lowering the viscosity of the blend.

### EXAMPLE 37

In preparing the sample, 8 grams of Eastman Vitamin E TPGS D-1000 were weighed into a PYREX® Media bottle and 2 grams of Nanopure water were then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. At this point 1.0 gram of tertiary-butyl hydroquinone (TBHQ, available from Eastman Chemical Company) was added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature and dark red in appearance. The post addition of TBHQ had no immediate effect on lowering the viscosity of the sample. After twenty-seven months the blend was free flowing.

### EXAMPLE 38

In preparing the sample, 8 grams of Eastman Vitamin E TPGS D-1000 were weighed into a PYREX® Media bottle and 2 grams of Nanopure water were then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. At this point 1.0 gram of diethyl phthalate, (DEP available from Eastman Chemical Company) was added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. The post addition of DEP had no immediate effect on lowering the viscosity of the sample. After twenty-seven months the sample was cloudy, flowing and highly viscous.

### EXAMPLE 39

In preparing the sample, 8 grams of Eastman Vitamin E TPGS D-1000 were weighed into a PYREX® Media bottle and 2 grams of Nanopure water were then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. At this point 1.0 gram of Grisiofulvin (available from Sigma Aldrich), was added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. The post addition of Grisiofulvin had no effect on lowering the viscosity of the sample.

### EXAMPLE 40

In preparing the sample, 8 grams of Eastman Vitamin E TPGS D-1000 were weighed into a PYREX® Media bottle and 2 grams of Nanopure water were then added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. At this point 1.0 gram of Vitamin E polyethylene glycol 400 succinate available from Eastman Chemical Company (TPGS 400) was added to the bottle. The bottle was sealed then placed in an oven. The oven temperature was set at 80° C. After 6 hours the sample was removed and mixed thoroughly using a vortexer. The sample was returned to the oven and after 18 hours the oven was turned off. The sample was allowed to cool to room temperature then removed. The blend was a gel at room temperature. The post addition of Vitamin E TPGS 400 had no effect on lowering the viscosity of the sample.

### EXAMPLE 41

In preparing the sample, 9 grams of Vitamin E TPGS D-1000 were weighed into a PYREX® Media bottle and 1 gram of TPGS 400 was then added to the bottle. The bottle was sealed then placed in an oven. The oven temperature was set at 80° C. After 6 hours the sample was removed and mixed thoroughly using a vortexer. The sample was returned to the oven. After 18 hours the oven was turned off and the sample was allowed to cool to room temperature. The sample was a white waxy solid at room temperature.

The following examples illustrate that the eutectic mixtures of the present invention not only remain substantially in the liquid phase over extended periods but surprisingly do not effect the amount of ibuprofen in its free acid form.

### EXAMPLE 42

After 36 months, the composition of Example 31 was evaluated to determine the amount of ibuprofen in its free acid form that was present in the sample. The ibuprofen analysis was conducted using reverse phase HPLC with ultra violet detection using an Agilent HP1100. This method utilizes a gradient system with a C18 column. The amount of ibuprofen in the sample was determined to be 10.0 weight %.

### EXAMPLE 43

After 36 months, the composition of Example 30 was evaluated to determine the amount of ibuprofen in its free acid form that was present in the sample. Following the analysis procedure of Example 42 above, the amount of ibuprofen in the sample was determined to be 10.3 weight %.

### EXAMPLE 44

In preparing the sample, 50 grams of Vitamin E TPGS D-1000 NF were weighed into a container, placed in an oven and heated to 60° C. After the TPGS became liquid, it was mixed to ensure homogeneity. Ten grams of acetyl salicylic acid were weighed into a PYREX® Media bottle. Ten grams of the heated, liquid TPGS were then added to the bottle. The bottle was sealed and placed in an oven. The oven temperature was set at 100° C. After six hours the sample was removed. There were two layers in the bottle. The bottom layer was white which appeared to be the acetyl salicylic acid. The top layer was TPGS in its liquid form. The sample was returned to the oven and the oven temperature was increased to 150° C. The sample was heated for an additional six hours then the oven was turned off. The sample remained free flowing after cooling to room temperature.

### EXAMPLE 45

In preparing the sample, 50 grams of Vitamin E TPGS D-1000 NF were placed in an oven and heated to 60° C. Once liquid, the TPGS was mixed to ensure homogeneity. Ten grams of acetaminophen were weighed into a PYREX® Media bottle. Ten grams of the liquid TPGS were then added to the bottle. The bottle was sealed and placed in an oven. The oven temperature was set to 100° C. After six hours the sample was removed and there were two layers in the bottle. The bottom layer was white which appeared to be acetaminophen. The top layer was TPGS in its liquid form. The sample was returned to the oven and the oven temperature was increased to 150° C. The sample was heated for five hours then the oven was turned off. The sample was a solid after cooling to room temperature.

### EXAMPLE 46

In preparing the sample, 50 grams of Vitamin E TPGS D-1000 NF were placed in an oven and heated to 60° C. Once liquid, the TPGS was mixed to ensure homogeneity. Four grams of acetaminophen were weighed into a PYREX® Media bottle. Sixteen grams of the liquid TPGS were then added to the bottle. The bottle was sealed and placed in an oven. The oven temperature was set to 140° C. After six hours the sample was removed and there were two layers in the bottle. The bottom layer was white which appeared to be acetaminophen. The top layer was TPGS in its liquid form. The sample was a solid after cooling to room temperature.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

What is claimed is:

1. A composition comprising:
a. from about 10 to less than about 90 weight % of a first component comprising a pharmaceutically acceptable substituted C₆-C₁₀ aryl compound wherein the aryl moiety includes a straight or branched moiety selected from the group consisting of C₁-C₁₂ alkyl, C₁-C₁₂ alkoxy, C₂-C₆ alkanoyloxy, hydroxy, carboxy, carboxy substituted C₁-C₁₂ alkyl and mixtures and isomers thereof; and
b. from greater than 10 to about 90 weight % of a second component comprising a water-soluble preparation of a fat-soluble vitamin, wherein the percentages are based on the total weight of the first and second components and the composition is an eutectic composition.

2. The composition of claim 1 wherein the straight or branched moiety is selected from the group consisting of C₁-C₆ alkyl, C₁-C₆ alkoxy, C₂-C₃ alkanoyloxy, hydroxy, carboxy, monocarboxy substituted C₁-C₆ alkyl, di-carboxy substituted C₁-C₆ alkyl and mixtures thereof.

3. The composition of claim 2 wherein the substituted C₆-C₁₀ aryl compound includes up to four straight or branched moieties independently selected from the group consisting of C₁-C₆ alkyl, C₁-C₆ alkoxy, C₂-C₃ alkanoyloxy, hydroxy, carboxy, monocarboxy substituted C₁-C₆ alkyl, di-carboxy substituted C₁-C₆ alkyl and mixtures thereof.

4. The composition of claim 2 wherein the substituted C₆-C₁₀ aryl compound includes up to three straight or branched moieties independently selected from the group consisting of C₁-C₆ alkyl, C₁-C₆ alkoxy, C₂-C₃ alkanoyloxy, hydroxy, carboxy, monocarboxy substituted C₁-C₆ alkyl, di-carboxy substituted C₁-C₆ alkyl and mixtures thereof.

5. The composition of claim 1 wherein the first component is 2-(4-isobutylphenyl) propionic acid.

6. The composition of claim 1 wherein the first component is butylated hyroxyanisole.

7. The composition of claim 1 wherein the first component is acetyl salicylic acid.

8. The composition of claim 1 wherein the first component is from about 20 to about 70 weight % and the second component is from 80 to 30 weight %, wherein the percentages are based on the total weight of the first and second components.

9. The composition of claim 1 wherein the first component is from about 30 to about 50 weight % and the second component is from about 50 to about 70 weight %, wherein the percentages are based on the total weight of the first and second components.

10. The composition of claim 1 wherein the first component is from about 40 to about 50 weight % and the second component is from about 60 to about 50 weight %, wherein the percentages are based on the total weight of the first and second components.

11. The composition of claim 1 further comprising from about 0.1 to less than 10 weight percent, based on the total weight of the eutectic composition, of a component selected from the group consisting of diphenhydramine hydrochloride, pseudoephedrine hydrochloride, polyethylene glycol, polypropylene glycol, polyoxyethylene glycerol trihydroxystearate, C₁₂-C₁₈ polyoxyethylene fatty alcohol ethers, polyoxyethylene stearate, C₁₂-C₁₈ polyoxyethylenesorbitan mono fatty acid esters, d-α-tocopheryl, or polyoxyethylene castor oil derivatives.

12. The composition of claim 1 wherein the second component is a tocopheryl acid esterified with polyethylene glycol, and wherein the polyethylene glycol has a molecular weight of from about 200 to about 20,000.

13. The composition of claim 12 wherein the polyethylene glycol has a molecular weight of from about 400 to about 1500.

14. The composition of claim 12 wherein the polyethylene glycol has a molecular weight of about 1000.

15. A composition of matter comprising:
a. from about 30 to about 50 weight % of a first component comprising a pharmaceutically acceptable substituted C₆-C₁₀ aryl compound wherein the aryl moiety includes up to four straight or branched moieties independently selected from the group consisting of C₁-C₆ alkyl, C₁-C₆ alkoxy, C₂-C₃ alkanoyloxy, hydroxy, carboxy, carboxy substituted C₁-C₆ alkyl and mixtures and isomers thereof; and
b. from about 70 to about 50 weight % of a second component comprising a tocopheryl acid esterified with polyethylene glycol, and wherein the polyethylene glycol has a molecular weight of from about 400 to about 1500, wherein the percentages are based on the total weight of the first and second components and the composition is an eutectic composition.

16. The composition of claim 15 wherein the first component is 2-(4-isobutylphenyl) propionic acid.

17. The composition of claim 15 wherein the first component is butylated hyroxyanisole.

18. The composition of claim 15 wherein the first component is acetyl salicylic acid.

19. The composition of claim 15 wherein the first component is from about 40 to about 50 weight % and the second component is from 60 to 50 weight %, wherein the weight percentages are based on the total weight of the first and second components.

20. The composition of claim 15 wherein the second component is Vitamin E polyethylene glycol 1000 succinate.

21. A liquid softgel formulation comprising:
a. from about 30 to about 50 weight % of a first component comprising a pharmaceutically acceptable substituted C₆-C₁₀ aryl compound wherein the aryl moiety includes up to three straight or branched moieties selected from the group consisting of C₁-C₆ alkyl, C₁-C₆ alkoxy, C₂-C₃ alkanoyloxy, hydroxy, carboxy, carboxy substituted C₁-C₆ alkyl and mixtures and isomers thereof; and
b. from about 70 to about 50 weight % of a second component comprising a tocopheryl acid esterified with polyethylene glycol, and wherein the polyethylene glycol has a molecular weight of from about 400 to about 1500, wherein the percentages are based on the total weight of the first and second components and the composition is an eutectic composition.

22. The composition of claim 21 wherein the first component is selected from the group consisting of 2-(4-isobutylphenyl)prop ionic acid, butylated hyroxyanisole, acetyl salicylic acid and mixtures thereof.

23. The composition of claim 21 wherein the second component is Vitamin E polyethylene glycol 1000 succinate.

24. A method for preparing an eutectic composition of comprising:
a. forming a mixture comprising: i) from about 10 to less than about 90 weight % of a first component comprising a pharmaceutically acceptable substituted C₆-C₁₀ aryl compound wherein the aryl moiety includes a straight or branched moiety selected from the group consisting of C₁-C₁₂ alkyl, C₁-C₁₂ alkoxy, C₂-C₆ alkanoyloxy, hydroxy, carboxy, carboxy substituted C₁-C₁₂ alkyl and mixtures and isomers thereof; and ii) from 10 to about 90 weight % of a second component comprising water-soluble preparation of a fat-soluble vitamin, wherein the percentages are based on the total weight of the first and second components;
b. heating the mixture to a predetermined temperature for a period of time that is sufficient to substantially melt or fluidize at least one of the components; and
c. mixing the mixture sufficiently form a substantially fluidic eutectic blend.

25. The process of claim 24 wherein the temperature is from about 40° C. to about 95° C., and the time is from about 1 minute to about 24 hours.

26. The process of claim 24 wherein the temperature is from about 50° C. to about 80° C.

27. The process of claim 24 wherein the mixture comprises from about 40 to about 50 weight % of the first component and from about 60 to about 50 weight % of the second component, wherein the percentages are based on the total weight of the first and second components.

28. The process of claim 24 wherein the first component is selected from the group consisting of consisting of 2-(4-isobutylphenyl)propionic acid, butylated hyroxyanisole, acetyl salicylic acid and mixtures thereof.

29. The process of claim 24 wherein the second component is Vitamin E polyethylene glycol 1000 succinate.

30. The process of claim 24 further comprising cooling the eutectic blend.
